SEQUENCE LISTING (66 sequences)

SEQ ID NO: 1 - protein, 4 aa, Artificial Sequence
  synthetic polypeptide
YLGA

SEQ ID NO: 2 - protein, 4 aa, Artificial Sequence
  synthetic polypeptide
YLGG

SEQ ID NO: 3 - protein, 4 aa, Artificial Sequence
  synthetic polypeptide
YLGL

SEQ ID NO: 4 - protein, 4 aa, Artificial Sequence
  synthetic polypeptide
YLGI

SEQ ID NO: 5 - protein, 4 aa, Artificial Sequence
  synthetic polypeptide
YLGT

SEQ ID NO: 6 - protein, 4 aa, Artificial Sequence
  synthetic polypeptide
YLAA

SEQ ID NO: 7 - protein, 4 aa, Artificial Sequence
  synthetic polypeptide
YLLA

SEQ ID NO: 8 - protein, 4 aa, Artificial Sequence
  synthetic polypeptide
YLIA

SEQ ID NO: 9 - protein, 4 aa, Artificial Sequence
  synthetic polypeptide
YDAA

SEQ ID NO: 10 - protein, 4 aa, Artificial Sequence
  synthetic polypeptide
YAGA

SEQ ID NO: 11 - protein, 4 aa, Artificial Sequence
  synthetic polypeptide
YVGA

SEQ ID NO: 12 - protein, 4 aa, Artificial Sequence
  synthetic polypeptide
YIGA

SEQ ID NO: 13 - protein, 4 aa, Artificial Sequence
  synthetic polypeptide
SLGA

SEQ ID NO: 14 - protein, 4 aa, Artificial Sequence
  synthetic polypeptide
TLGA

SEQ ID NO: 15 - protein, 4 aa, Artificial Sequence
  synthetic polypeptide
FLGA

SEQ ID NO: 16 - protein, 4 aa, Artificial Sequence
  synthetic polypeptide
WLGA

SEQ ID NO: 17 - protein, 4 aa, Artificial Sequence
  synthetic polypeptide
DLGA

SEQ ID NO: 18 - protein, 4 aa, Artificial Sequence
  synthetic polypeptide
ELGA

SEQ ID NO: 19 - protein, 6 aa, Artificial Sequence
  synthetic polypeptide
YLGAVF

SEQ ID NO: 20 - protein, 5 aa, Artificial Sequence
  synthetic polypeptide
SYLGA

SEQ ID NO: 21 - protein, 6 aa, Artificial Sequence
  synthetic polypeptide
SSYLGA

SEQ ID NO: 22 - protein, 7 aa, Artificial Sequence
  synthetic polypeptide
RSSYLGA

SEQ ID NO: 23 - protein, 9 aa, Artificial Sequence
  synthetic polypeptide
RSSYLGAVF

SEQ ID NO: 24 - protein, 6 aa, Artificial Sequence
  synthetic polypeptide
YLGGVR

SEQ ID NO: 25 - protein, 5 aa, Artificial Sequence
  synthetic polypeptide
IYLGG

SEQ ID NO: 26 - protein, 6 aa, Artificial Sequence
  synthetic polypeptide
PIYLGG

SEQ ID NO: 27 - protein, 7 aa, Artificial Sequence
  synthetic polypeptide
EPIYLGG

SEQ ID NO: 28 - protein, 9 aa, Artificial Sequence
  synthetic polypeptide
EPIYLGGVF

SEQ ID NO: 29 - protein, 12 aa, Artificial Sequence
  synthetic polypeptide
HHIYLGATNYIY

SEQ ID NO: 30 - protein, 11 aa, Artificial Sequence
  synthetic polypeptide
HIYLGATNYIY

SEQ ID NO: 31 - protein, 10 aa, Artificial Sequence
  synthetic polypeptide
IYLGATNYIY

SEQ ID NO: 32 - protein, 9 aa, Artificial Sequence
  synthetic polypeptide
YLGATNYIY

SEQ ID NO: 33 - protein, 11 aa, Artificial Sequence
  synthetic polypeptide
HHIYLGATNYI

SEQ ID NO: 34 - protein, 10 aa, Artificial Sequence
  synthetic polypeptide
HHIYLGATNY

SEQ ID NO: 35 - protein, 9 aa, Artificial Sequence
  synthetic polypeptide
HHIYLGATN

SEQ ID NO: 36 - protein, 8 aa, Artificial Sequence
  synthetic polypeptide
HHIYLGAT

SEQ ID NO: 37 - protein, 7 aa, Artificial Sequence
  synthetic polypeptide
HHIYLGA

SEQ ID NO: 38 - protein, 30 aa, Artificial Sequence
  synthetic polypeptide
PIQNVVLHKHHIYLGATNYIYVLNDKDLQK

SEQ ID NO: 39 - protein, 30 aa, Artificial Sequence
  synthetic polypeptide
YCTTGQMWARSSYLGAVFNLTSADHLYVNV

SEQ ID NO: 40 - protein, 4 aa, Artificial Sequence
  synthetic polypeptide
LLGA

SEQ ID NO: 41 - protein, 4 aa, Artificial Sequence
  synthetic polypeptide
ILGA

SEQ ID NO: 42 - protein, 4 aa, Artificial Sequence
  synthetic polypeptide
ALGA

SEQ ID NO: 43 - protein, 4 aa, Artificial Sequence
  synthetic polypeptide
GLGA

SEQ ID NO: 44 - protein, 4 aa, Artificial Sequence
  synthetic polypeptide
YGGA

SEQ ID NO: 45 - protein, 4 aa, Artificial Sequence
  synthetic polypeptide
YFGA

SEQ ID NO: 46 - protein, 4 aa, Artificial Sequence
  synthetic polypeptide
YTGA

SEQ ID NO: 47 - protein, 4 aa, Artificial Sequence
  synthetic polypeptide
KLGA

SEQ ID NO: 48 - protein, 55 aa, Homo sapiens
ETGKTNYNTCYTAGVCLLKARQKIAVKMVHADISINMSKHTTFFGAIRLGEAPAS

SEQ ID NO: 49 - protein, 55 aa, Homo sapiens; identical to SEQ ID NO: 48

SEQ ID NO: 50 - protein, 49 aa, Homo sapiens
SSAKPCGQQSIHLGGVFELQPGASVFVNVTDPSQVSHGTGFTSFGLLKL

SEQ ID NO: 51 - protein, 49 aa, Homo sapiens
CTTGQMWARSSYLGAVFNLTSADHLYVNVSELSLVNFEESQTFFGLYKL

SEQ ID NO: 52 - protein, 45 aa, Homo sapiens
MVASLTYKDKVYLNVTTDNTSLDDFHVNGGELILIHQNPGEFCVL

SEQ ID NO: 53 - protein, 47 aa, Homo sapiens
SKIQNVGGTYELHVGDTIDLIFNSEHQVLKNNTYWGIILLANPQFIS

SEQ ID NO: 54 - protein, 58 aa, Homo sapiens
CESGMQTKHVYQNLSQFLLDYLQVNTTISVNVDTFQYIDTSTFPLENVLSIFLYSNSD

SEQ ID NO: 55 - protein, 50 aa, Homo sapiens
CEVGSNWFQPIYLGAMFSLQEGDKLMVNVSDISLVDYTKEDKTFFGAFLL

SEQ ID NO: 56 - protein, 54 aa, Homo sapiens
CGRATSSSRVWWDSSFLGGVVHLEAGEEVVVRVLDERLVRLRDGTRSYFGAFMV

SEQ ID NO: 57 - protein, 66 aa, Homo sapiens
SSEARNSAFGFQGRLLHLSAGQRLGVHLHTEARARHAWQLTQGATVLGLFRVTPEIPAGLPSPRSE

SEQ ID NO: 58 - protein, 48 aa, Homo sapiens
SFHQGCTIASQRLTPLARGDTLCTNLTGTLLPSRNTDETFFGVQWVRP

SEQ ID NO: 59 - protein, 50 aa, Homo sapiens
YPGLQEPWLHSMYHGAAFQLTQGDQLSTHTDGIPHLVLSPSTVFFGAFAL

SEQ ID NO: 60 - protein, 57 aa, Homo sapiens
CQRETPEGAEAKPWYEPIYLGGVFQLEKGDRLSAEINRPDYLDFAESGQVYFGIIAL

SEQ ID NO: 61 - protein, 59 aa, Homo sapiens
TPVLDPARRQGYGPLWYTSVGFGGLVQLRRGERVYVNISHPDMVDFARGKTFFGAVMVG

SEQ ID NO: 62 - protein, 54 aa, Homo sapiens
SATAASSLGPQLRLCQVSGLLALRPGSSLRIRTLPWAHLKAAPFLTYFGLFQVH

SEQ ID NO: 63 - protein, 50 aa, Homo sapiens
PSHPDRAYNSCYSAGVFHLHQGDILSVIIPRARAKLNLSPHGTFLGFVKL

SEQ ID NO: 64 - protein, 49 aa, Homo sapiens
PETLPNNSCYSAGIAKLEEGDELQLAIPRENAQISLDGDVTFFGALKLL

SEQ ID NO: 65 - protein, 55 aa, Homo sapiens
YWSGNSEFHFYSINVGGFFKLRSGEEISIEVSNPSLLDPDQDATYFGAFKVRDID

SEQ ID NO: 66 - protein, 52 aa, Homo sapiens
CWSKDAEYGLYSIYQGGIFELKENDRIFVSVTNEHLIDMDHEASFFGAFLVG

(19) United States
(12) Patent Application Publication
(10) Pub. No.: US 2008/0280834 A1
(43) Pub. Date: Nov. 13, 2008

(54) Compounds and methods for inhibiting apoptosis

(75) Inventors: Reza Zarnegar, Gibsonia, PA (US); Marie C. DeFrances, Gibsonia, PA (US); Trina L. Foster, Ottawa (CA); George L. McLendon, Durham, NC (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignees: University of Pittsburgh of the Commonwealth System of Higher Education, Pittsburgh, PA (US); Duke University, Durham, NC (US)

(21) Appl. No.: 11/607,396

(22) Filed: Dec. 1, 2006

Related U.S. Application Data
(60) Provisional application No. 60/741,125, filed on Dec. 1, 2005.
(60) Provisional application No. 60/750,888, filed on Dec. 16, 2005.

(51) Int. Cl. A61K 38/07; C07K 5/00; C07K 7/06; A61K 38/08; A61P 35/00

(52) U.S. Cl. 514/16; 530/330; 514/18; 530/328

(57) ABSTRACT

The invention provides compounds, compositions, and methods for inhibiting apoptosis and for the treatment of conditions related thereto.

### CROSS-REFERENCE TO RELATED APPLICATION

This patent application claims the benefit of U.S. Provisional Patent Application No. 60/741,125, filed Dec. 1, 2005, and U.S. Provisional Patent Application No. 60/750,888, filed Dec. 16, 2005, each of which is incorporated by reference in its entirety.

### STATEMENT REGARDING FEDERALLY SPONSORED RESEARCH AND DEVELOPMENT

This invention was made in part with Government support under Grant Number GM 059348-05 A1 awarded by the National Institutes of Health. The Government may have certain rights in this invention.

### BACKGROUND OF THE INVENTION

Apoptosis (programmed cell death) plays a central role in the development and homeostasis of all multi-cellular organisms. Alterations in apoptotic pathways have been implicated in many types of human pathologies, including developmental disorders, cancer, autoimmune diseases, as well as neuro-degenerative disorders. It is a tightly regulated pathway governing the death processes of individual cells and can be initiated either extrinsically or intrinsically. The former is an intracellular mechanism triggered by the mitochondria while the latter involves the interaction of a ‘death receptor’ with its corresponding ligand at the cell membrane.

Thus, the programmed cell death pathways have become attractive targets for development of therapeutic agents. In particular, since it is conceptually easier to kill cells than to sustain cells, attention has been focused on anti-cancer therapies using pro-apoptotic agents such as conventional radiation and chemotherapy. These treatments are generally believed to trigger activation of the mitochondria-mediated apoptotic pathways. However, these therapies lack molecular specificity, and more specific molecular targets are needed.

### BRIEF SUMMARY OF THE INVENTION

The present invention provides peptides, peptidomimetics and methods of their use for inhibiting or otherwise regulating apoptosis. Compositions comprising the inventive compounds are also provided by the invention. Methods of inhibiting apoptosis and treating TNFR associated conditions and diseases utilizing such compositions are provided herein as well.

These aspects and additional inventive features will be apparent upon reading the following detailed description and reviewing the accompanying drawing.

### BRIEF DESCRIPTION OF THE DRAWING

The FIGURE depicts the sequence alignment of proteins discussed herein.

### DETAILED DESCRIPTION

In a first aspect, the present invention provides peptides and peptidomimetics. In one embodiment of the present invention the peptide or peptidomimetic comprises the general formula I.

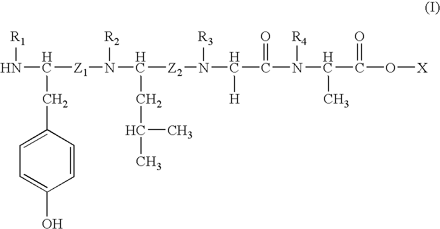

wherein Z₁ and Z₂ are independently selected from C═O and CH₂; R₁ is selected from a H, a lower alkyl (such as from 1 to 10 carbon atoms), optionally a substituted lower alkyl, a carboxylic acid and an extended peptide up to three residues long selected from H—H—I, R—S—S, and E-P—I; R₂, is selected from a H, a lower alkyl (such as from 1 to 10 carbon atoms), optionally a substituted lower alkyl, and a carboxylic acid; R₃, and R₄ are H, and X is selected from COOH and CONH₂.

In another embodiment, the invention provides a compound comprised of a peptidomimetic of a tetrapeptide having the formula IV.

In another embodiment, the invention provides a peptidomimetic of a peptide of the sequence X1-X2-X3-X4 wherein X1 is an amino acid selected from Y, S, T, F, W, D and E; X2 is an amino acid selected from L, A, V, I, G, F, and T; X3 is an amino acid selected from G, A, L, I and A; and X4 is an amino acid selected from A, G, L, I, and T is provided.

In another embodiment, the invention provides a compound comprising the formula X1-X2-X3-X4 wherein XI is an amino acid selected from Y, S, T, F, W, D or E or a mimetic of an amino acid selected from Y, S, T, F, W, D, or E; X2 is an amino acid selected from L, A, V, I, G, F, or T or a mimetic of an amino acid selected from L, A, V, I, G, F, or T; X3 is an amino acid selected from G, A, L, I or A or a mimetic of an amino acid selected from G, A, L, I, or A; and X4 is A, G, L, I, or T or a mimetic of an amino acid selected from A, G, L, I or T.

In another embodiment, the invention provides a peptidomimetic of a tetrapeptide, the tetrapeptide comprising the sequence X1-X2-X3-X4 wherein X1 is an amino acid selected from Y, S, T, F, W, D and E; X2 is an amino acid selected from L, A, V, I, G, F, and T; X3 is an amino acid selected from G, A, L, I and A; and X4 is an amino acid selected from A, G, L, I, and T.

In another embodiment, the invention provides a compound comprising the formula X1-X2-X3-X4-X5-X6-X7-X8 wherein X1 is an amino acid selected from Y, S, T, F, W, D or E or a mimetic of an amino acid selected from Y, S, T, F, W, D, or E; X2 is a spacer; X3 is an amino acid selected from L, A, V, I, G, F, or T or a mimetic of an amino acid selected from L, A, V, I, G, F, or T; X4 is a spacer; X5 is an amino acid selected from G, A, L, I or A or a mimetic of an amino acid selected from G, A, L, I, or A; X6 is a spacer; X7 is an amino acid selected from A, G, L, I, or T or a mimetic of an amino acid selected from A, G, L, I or T; and X8 is a spacer. Optionally, the formula may include X0, such that the formula comprises X0-X1-X2-X3-X4-X5-X6-X7-X8, wherein X1-X8 are as previously defined, and X0 is a peptide of the general formula A1-A2-A3, where A1 is a charged amino acid selected from H, K, R, E, and D or a mimetic of H, K, R, E, and D; A2 is an amino acid selected from S, A, and T or a mimetic of S, A, and T; and A2 is an amino acid selected from S, A, T, I, and V, or a mimetic of S, A, T, I, and V.

In another embodiment, the invention provides a compound comprising X1-X2-X3-X4-X5-X6-X7-X8 wherein X1 is a spacer; X2 is an amino acid selected from Y, S, T, F, W, D or E or a mimetic of an amino acid selected from Y, S, T, F, W, D, or E; X3 is a spacer; X4 is an amino acid selected from L, A, V, I, G, F, or T or a mimetic of an amino acid selected from L, A, V, I, G, F, or T; X5 is a spacer; X6 is an amino acid selected from G, A, L, I or A or a mimetic of an amino acid selected from G, A, L, I, or A; X7 is a spacer; and X8 is an amino acid selected from A, G, L, I, or T or a mimetic of an amino acid selected from A, G, L, I or T.

In another embodiment, the invention provides a compound comprising the formula II.

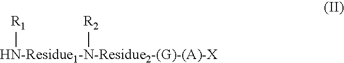

wherein Residue₁ is an amino acid selected from Y, S, T, D, and E; Residue₂ is an amino acid selected from A, V, L, I, M and a lower alkyl (such as from 1 to 5 carbon atoms), optionally a substituted lower alkyl; R₁ and R₂ are independently selected from H, a lower allyl (such as from 1 to 10 carbon atoms), optionally a substituted lower allyl, and carboxylic acid; and X is selected from COOH and CONH₂ is provided.

Some exemplary inventive compounds include YLGA (SEQ ID NO: 1), YLGG (SEQ ID NO:2), YLGL (SEQ ID NO:3), YLGI (SEQ ID NO:4), YLGT (SEQ ID NO:5), YLAA (SEQ ID NO:6), YLLA (SEQ ID NO:7), YLIA (SEQ ID NO:8), Y1dAA (SEQ ID NO:9), YAGA (SEQ ID NO:10), YVGA (SEQ ID NO:11), YIGA (SEQ ID NO:12), SLGA (SEQ ID NO:13), TLGA (SEQ ID NO:14), FLGA (SEQ ID NO:15), WLGA (SEQ ID NO:16), DLGA (SEQ ID NO:17), ELGA (SEQ ID NO:18), YLGAVF (SEQ ID NO:19), SYLGA (SEQ ID NO:20), SSYLGA (SEQ ID NO:21), RSSYLGA (SEQ ID NO:22), RSSYLGAVF (SEQ ID NO:23), YLGGVR (SEQ ID NO:24), IYLGG (SEQ ID NO:25), PIYLGG (SEQ ID NO:26), EPIYLGG (SEQ ID NO:27), EPIYLGGVF (SEQ ID NO:28), HHIYLGATNYIY (SEQ ID NO:29), HIYLGATNYIY (SEQ ID NO:30), IYLGATNYIY (SEQ ID NO:31), YLGATNYIY (SEQ ID NO:32), HHIYLGATNYI (SEQ ID NO:33), HHIYLGATNY (SEQ ID NO:34), HHIYLGATN (SEQ ID NO:35), HHIYLGAT (SEQ ID NO:36), HHIYLGA (SEQ ID NO:37), and peptidomimetics thereof.

Where one or more chiral centers exist in an amino acid, artificial amino acid, or atom of an inventive compound any of the enantiomers, D or L and more generally R or S configuration, or diastereoisomers may optionally be used.

The inventive compounds may be prepared by any suitable peptide synthesis technique, such as, for example, liquid-phase synthesis or solid-phase peptide synthesis (SPPS). Such techniques are well known in the art. A preferred method of peptide synthesis is SPPS, which allows the synthesis of peptides comprising natural and non-naturally occurring amino acids. In SPPS, small beads are treated with linkers on which peptide chains may be built. The synthesis beads retain strong bondage to the peptides until cleaved by a reagent such as trifluoroacetic acid. The beads create a synthesis environment in which the peptide chains being created will not pass through a filter material while the reagents used to create them will. The two most common forms of SPPS are Fmoc (which uses a fluorenyl-methoxy-carbonyl protecting group) and Bmoc (which uses a tert-butyloxy-carbonyl protecting group). There are automated synthesizers available for both techniques, or the techniques may be performed manually.

As noted, in some embodiments, the inventive compound is a peptidomimetic. peptidomimetics are synthetic compounds having a three-dimensional structure (i.e. a “core peptide motif”) based upon the three-dimensional structure of a selected peptide. The peptide motif provides the mimetic compound with the desired biological activity (e.g., binding to FasR and/or TNFR) wherein the binding activity of the mimetic compound is not substantially reduced, and is often the same as or greater than the activity of the native peptide on which the mimetic is modeled. Peptidomimetic compounds can have additional characteristics that enhance their therapeutic application, such as increased cell permeability, greater affinity and/or avidity and prolonged biological half-life.

The terms “mimetic,” “peptide mimetic,” “modified peptide,” and, “peptidomimetic” are used interchangeably herein and generally refer to a peptide, partial peptide or non-peptide molecule that mimics the tertiary binding structure or activity of a selected native peptide or protein functional domain. A dimer of this compound is a molecule which mimics the tertiary structure or activity in two separate and distinct regions of the subject molecule. These peptide mimetics can include chemically modified amino acids or peptides, as well as non-peptide agents such as small molecule drug mimetics.

Mimetic, specifically, peptidomimetic design strategies are readily available in the art (see, e.g., Ripka & Rich, Curr. Op. Chem. Biol. 2, 441-452, 1998; Hruby et al., Curr. Op. Chem. Biol. 1, 114-119, 1997; Hruby & Balse, Curr. Med. Chem. 9, 945-970, 2000). One class of mimetic mimics a backbone that is partially or completely non-peptide, but mimics the peptide backbone atom-for-atom and comprises side groups that likewise mimic the functionality of the side groups of the native amino acid residues. Several types of chemical bonds, e.g. ester, thioester, thioamide, retroamide, reduced carbonyl, dimethylene and ketomethylene bonds, are known in the art to be generally useful substitutes for peptide bonds in the construction of protease-resistant peptidomimetics. Another class of peptidomimetics comprises a small non-peptide molecule that binds to another peptide or protein, but which is not necessarily a structural mimetic of the native peptide. Yet another class of peptidomimetics has arisen from combinatorial chemistry and the generation of massive chemical libraries. These generally comprise novel templates which, though structurally unrelated to the native peptide, possess necessary functional groups positioned on a nonpeptide scaffold to serve as “topographical” mimetics of the original peptide (Ripka & Rich, 1998, supra).

To produce peptidomimetics, peptides in the inventive compounds of the invention can be modified to produce peptide mimetics by replacement of one or more naturally occurring side chains of the 20 genetically encoded amino acids, or D amino acids with other side chains, for instance with groups such as alkyl, lower alkyl, cyclic 4-, 5-, 6-, to 7-membered alkyl, amide, amide lower alkyl, amide di(lower alkyl), lower alkoxy, hydroxy, carboxy and the lower ester derivatives thereof, and with 4-, 5-, 6-, to 7-membered heterocyclics. Such modifications can occur after peptide synthesis or before peptide synthesis (i.e., involving synthesis of the polypeptide using modified amino acids). For example, proline analogs can be made in which the ring size of the proline residue is changed from 5 members to 4, 6, or 7 members. Cyclic groups can be saturated or unsaturated, and if unsaturated, can be aromatic or non-aromatic. Heterocyclic groups can contain one or more nitrogen, oxygen, and/or sulphur heteroatoms. Examples of such groups include the furazanyl, imidazolidinyl, imidazolyl, imidazolinyl, isothiazolyl, isoxazolyl, morpholinyl (e.g. morpholino), oxazolyl, piperazinyl (e.g. 1-piperazinyl), piperidyl (e.g. 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g. 1-pyrrolidinyl), pyrrolinyl, pyrrolyl, thiadiazolyl, thiazolyl, thienyl, thiomorpholinyl (e.g. thiomorpholino), and triazolyl. These heterocyclic groups can be substituted or unsubstituted. Where a group is substituted, the substituent can be alkyl, alkoxy, halogen, oxygen, or substituted or unsubstituted phenyl. Peptidomimetics may also have amino acid residues that have been chemically modified by phosphorylation, sulfonation, biotinylation, or the addition or removal of other moieties.

The term “heteroatom” refers to nitrogen, oxygen, sulfur, other atoms or groups where the nitrogen, sulfur and other atoms may optionally be oxidized, and the nitrogen may optionally be quaternized. Any heteroatom with unsatisfied valences is assumed to have hydrogen atoms sufficient to satisfy the valences. In some embodiments, for example where the heteroatom is nitrogen and includes a hydrogen or other group to satisfy the valance of the nitrogen atom, replacement of the nitrogen in a similar structure by another heteroatom, for example by oxygen, will result in the hydrogen or group previously bonded to the nitrogen to be absent. The term heteroatom may include but is not limited to for example —O—, —S—, —S(O)—, —S(O)₂—, —N—, —N(H)—, and —N(C₁-C₆ alkyl).

The term “alkyl” refers to a saturated straight, branched, or cyclic hydrocarbon having from about 1 to about 30 carbon atoms (and all combinations and subcombinations of ranges and specific numbers of carbon atoms therein). Lower alkyl group refers to a saturated straight, branched, or cyclic hydrocarbon having group of 1 to 10 carbon atoms, suitably 1 to 5 carbon atoms and all combinations and subcombinations of ranges and specific numbers of carbon atoms therein. Alkyl groups include, but are not limited to methyl, ethyl, n-propyl, isopropyl, n-butyl, isobutyl, t-butyl, n-pentyl, cyclopropyl, methylcyclopropyl, cyclopentyl, isopentyl, neopentyl, n-hexyl, isohexyl, cyclohexyl, cyclooctyl, adamantyl, 3-methylpentyl, 2,2-dimethylbutyl, and 2,3-dimethylbutyl.

The term “substituted allyl” refers to a saturated straight, branched, or cyclic hydrocarbon having from about 1 to about 30 carbon atoms (and all combinations and subcombinations of ranges and specific numbers of carbon atoms therein) having from 1 to 5 substituents. Substituted lower alkyl group refers to a saturated straight, branched, or cyclic hydrocarbon of 1 to 10 carbon atoms, suitably 1 to 5 carbon atoms (and all combinations and subcombinations of ranges and specific numbers of carbon atoms therein) having from 1 to 5 substituents. Substituted alkyl radicals and substituted lower alkyl groups can have from 1 to 5 substituents including but not limited to alkoxy, substituted alkoxy, acylamino, thiocarbonylamino, acyloxy, amino, amidino, alkylamidino, amidalkyl (such as —CH₂C(═O)NH₂ or —CH₂CH₂C(═O)NH₂), thioamidino, acylalkylamino, cyano, halogen atoms (F, Cl, Br, I) to give halogenated or partially halogenated alkyl groups, including but not limited to —CF₃, —CF₂CF₃, —CH₂CF₂Cl and the like, hydroxy, nitro, carboxyl, carboxylalkyl, carboxylheterocyclic, carboxyl-substituted heterocyclic, cycloalkyl, guanidino, heteroaryl, aryl, heterocyclic, alkylamino, dialkylamino, or optionally substituted versions of any of the aforementioned groups.

The term “alkylene radical” as used herein includes reference to a di-functional saturated branched or unbranched hydrocarbon radical containing from 1 to 30 carbon atoms, and includes, for example, methylene (—CH₂—), ethylene (—CH₂CH₂—), propylene (—CH₂CH₂CH₂—), 2-methylpropylene (—CH₂CH(CH₃)CH₂—), hexylene (—(CH₂)₆—), and the like. Lower allylene includes an alkylene group of 1 to 10, suitably 1 to 5, carbon atoms.

Substituted alkylene radicals includes reference to a di-functional saturated branched or unbranched allylene radical or group having 1-30 carbon atoms and having from 1 to 5 substituents. Lower substituted allylene radicals refer to a substituted alkylene radical group, having 1-10 carbon atoms, suitably having 1-5 carbon atoms, and having from 1 to 5 substituents. Substituents can include but are not limited to those for the allyl groups.

The term “alkenyl radical” as used herein includes reference to a branched, cyclic hydrocarbon, or unbranched hydrocarbon radical of 2 to 30 carbon atoms containing at least one carbon-carbon double bond, such as ethenyl, n-propenyl, isopropenyl, n-butenyl, isobutenyl, t-butenyl, octenyl, decenyl, tetradecenyl, hexadecenyl, eicosenyl, tetracosenyl and the like. The term lower alkenyl includes an alkenyl group of 2 to 10 carbon atoms, suitably 2 to 5 carbon atoms, containing at least one carbon-carbon double bond. The one or more carbon-carbon double bonds may independently have a cis or trans configuration. Substituted alkenyl radical refers to an alkenyl radical or lower alkenyl group having from 1 to 5 substituents that can include but are not limited to those for the alkyl groups.

The term “alkenylene radical” includes reference to a difunctional branched or unbranched hydrocarbon radical or group containing from 2 to 30 carbon atoms and at least one carbon-carbon double bond. “Lower alkenylene” includes an alkenylene group of 2 to 10, suitably 2 to 5, carbon atoms, containing one carbon-carbon double bond. Substituted alkenylene radical refers to an alkenylene radical or lower alkenyl group having from 1 to 5 substituents that can include but are not limited to those for the alkyl groups.

The term “alkynyl radical” or group refers to straight or branched chain hydrocarbon radical having 2 to 12 carbon atoms and at least one triple bond, some embodiments include alkynyl groups of 2 to 6 carbon atoms that have one triple bond. A substituted alkynyl will contain one, two, or three substituents as defined for substituted alkyl groups. Alkynylene includes reference to a difunctional branched or unbranched hydrocarbon chain containing from 2 to 12 carbon atoms and at least one carbon-carbon triple bond; some embodiments include an alkynylene groups of 2 to 6 carbon atoms with one triple bond. A substituted alkynylene will contain one, two, or three substituents as defined for substituted alkyl groups.

As used herein, “halo” or halogen refers to any halogen, such as I, Br, Cl or F. As used herein, “cyano” refers to the —C═N group.

The term “aryl radical” or group refers to an optionally substituted, mono or bicyclic aromatic ring radicals having from about 5 to about 14 carbon atoms (and all combinations and subcombinations of ranges and specific numbers of carbon atoms therein), with from about 6 to about 10 carbons being preferred. Non-limiting examples or aryl groups include, for example, phenyl and naphthyl. A substituted aryl group will contain one or more substituents as defined for substituted alkyl groups.

“Aralkyl radical” refers to alkyl radicals bearing an aryl substituent and have from about 6 to about 20 carbon atoms (and all combinations and subcombinations of ranges and specific numbers of carbon atoms therein), with from about 6 to about 12 carbon atoms being preferred. Aralkyl groups can be optionally substituted. Non-limiting examples include, for example, benzyl, naphthylmethyl, diphenylmethyl, triphenylmethyl, phenylethyl, and diphenylethyl. A substituted arylalkyl group will contain one or more substituents on the aryl or allyl group as defined for substituted alkyl groups.

“Cycloalkylaryl radical” or group refers to a cycloalkyl radical fused to an aryl group, including all combinations of independently substituted alkyl cycloalkylaryls, the cycloalkyl and aryl group having two atoms in common. Examples of fused cycloalkylaryl groups used in compounds may include 1-indanyl, 2-indanyl, 1-(1,2,3,4-tetrahydronaphthyl), and the like. Tetrahydronaphthyl more specifically refers to those univalent radicals or groups derived from fused polycyclic hydrocarbons including all combinations of independently substituted allyl tetrahydronaphthyls. These radicals may have a point of attachment at (C₁) or equivalently (C₄) in structure (III), or position labeled (C₂) and equivalently (C₃) in structure (IIIa). The chiral carbon atoms C₁₋₄ in tetrahydronaphthalene and its alkyl substituted derivatives may have either an (R) or (S) configuration.

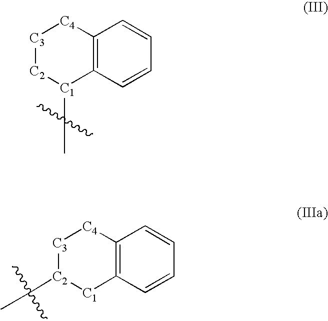

“Cycloalkyl radical” or group more specifically includes reference to a monovalent saturated carbocyclic alkyl radical consisting of one or more rings in their structures and having from about 3 to about 14 carbon atoms (and all combinations and subcombinations of ranges and specific numbers of carbon atoms therein), with from about 3 to about 7 carbon atoms being preferred. Multi-ring structures may be bridged or fused ring structures. The rings can optionally be substituted with one or more of the substituents for the allyl groups. Examples of cycloalkyl groups include, but are not limited to, cyclopropyl, cyclobutyl, cyclopentyl, cyclohexyl, cyclooctyl, and adamantyl. A substituted cycloalkyl group will contain one or more substituents as defined for substituted alkyl groups.

“Cycloalkylalkyl radical” more specifically refers to alkyl radicals bearing an cycloalkyl substituent and having from about 4 to about 20 carbon atoms (and all combinations and subcombinations of ranges and specific numbers of carbon atoms therein), with from about 6 to about 12 carbon atoms being preferred and can include but are not limited to methyl-cyclopropyl, methylcyclohexyl, isopropylcyclohexyl, and butyl-cyclohexyl groups. Cycloalkylalkyl radical or group can be optionally substituted with one or more substituents for the alkyl groups including but not limited to hydroxy, cyano, allyl, alkoxy, thioalkyl, halo, haloalkyl, hydroxyalkyl, nitro, amino, alkylamino and dialkylamino.

The term “acyl” refers to an alkyl, substituted alkyl, alkenyl, alkynyl, cycloalkyl, cycloalkenyl, aryl, heteroaryl, or heterocyclyl group as defined above bonded through one or more carbonyl —C(═O)— groups to give a group of formula —C(═O)R where R is the substituted alkyl, alkenyl, alkynyl, cycloalkyl, cycloalkenyl, aryl, heteroaryl, or heterocyclyl group. When the term acyl is used in conjunction with another group, as in acylamino, this refers to the carbonyl group {—C(═O)} linked to the second named group. Thus, acylamino or carbamoyl refers to —C(═O)NH₂, acylalkylamino can refer to groups such as —C(═O)NR′R″ where R′ and R″ can be H or alkyl. Amidalkyl refers to groups such as —CH₂C(═O)NH₂, —CH₂CH₂C(═O)NH₂) and more generally —(CH₂)ₚC(═O)NH₂. Carboxy refers to the radical or group —C(═O)OH, carboxyalkyl refers to the groups such as —(CH₂)ₚC(═O)OH, allyl carboxyalkyl refers to groups such as (—C(═O)O-(alkyl)), and alkoxycarbonyl or acylalkoxy refers to a (—C(═O)O-(alkyl)) group, where allyl is previously defined. As used herein aryoyl or acylaryl refers to a (—C(═O)(aryl)) group, wherein aryl is as previously defined. Exemplary aroyl groups include benzoyl and naphthoyl. Acetyl refers to the group CH₃C(═O)—. Formyl refers to the radical or group HC(═O)—. In the aforementioned group p can be independently the integer 0, 1, 2, or 3.

“Aryloxy radical” refers to optionally substituted mono or bicyclic aromatic radical having from about 5 to about 14 carbon atoms and an (aryl-O—) radical group wherein aryl is as previously defined. Such aryloxy radicals include but are not limited to that illustrated by the radical of formula (IV). Optional substituents on the aryl ring in the aryloxy radical may include but are not limited to hydrogen, alkyl, halogen, hydroxy, alkoxy, alkoxycarbonyl or other substituents. Embodiments of IAP binding compounds of the present invention can include an optionally aryloxy group like the phenoxy radical linked to the pyrrolidine ring.

The terms “alkoxy” and “alkoxyl” refer to an optionally substituted (alkyl-O—) radical or group wherein alkyl is as previously defined. Exemplary alkoxy radicals or groups include methoxy, ethoxy, n-propoxy, i-propoxy, n-butoxy, cyclopropyl-methoxy, and heptoxy. Alkoxy radicals can also include optionally substituted alkyl in the alkylO-group. Alkoxy can include including optionally substituted aryl groups as previously defined and illustrated by the non-limiting radical of formula (V). A “lower alkoxy” group refers to an optionally substituted alkoxy group containing from one to five carbon atoms. “Polyether” refers to a compound or moiety possessing multiple ether linkages, such as, but not limited to, polyethylene glycols or polypropylene glycols. “Polyalkylethers” refers to alkyls interconnected by or otherwise possessing multiple ether linkages. “Arylalkyloxy” means an arylalkyl-O— group in which the arylalkyl group is as previously described. Exemplary arylalkyloxy groups include benzyloxy (C₆H₅CH₂O—) radical (BnO—), or 1- or 2-naphthalenemethoxy. Optional substituents on the aryl ring in the benzyloxy radical may include but are not limited to hydrogen, alkyl, halogen, hydroxy, alkoxy, and alkoxycarbonyl or other substituents as defined for the alkyl group.

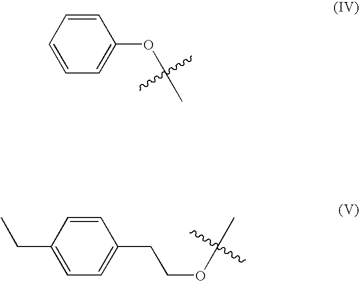

“Arylamino radical” refers to optionally substituted mono or bicyclic aromatic radical having from about 5 to about 14 carbon atoms and an (—NH(aryl)) radical group wherein aryl can be optionally substituted as previously defined for alkyl. Optional substituents on the aryl ring in the arylamino radical may include but are not limited to hydrogen, alkyl, halogen, hydroxy, alkoxy, and alkoxycarbonyl. An example of an arylamino group is the anilino radical or group. Amino refers to an —NH₂ group and alkylamino refer to a radical (—NH R′) group wherein R′ is H, alkyl, cycloalkyl, aryl, heteroaryl, aralkyl or optionally substituted versions of these as previously defined. Exemplary alkylamino radical groups include methylamino, ethylamino, n-propylamino, i-propylamino, n-butylamino, and heptylamino. The benzylamino radical refers to the arylamino group C₆HsCH₂NH—, the aryl group may have optional substituents including but are not limited to hydrogen, alkyl, halogen, hydroxy, alkoxy, and alkoxycarbonyl or other substituents.

“Dialkylamino” includes reference to a radical (—NR′R″), wherein R′ and R″ can be each independently be an H, alkyl, cycloalkyl, aryl, heteroaryl, aralkyl or optionally substituted versions of these as previously defined. Examples of dialkylamino radicals include, but are not limited to, dimethylamino, methylethylamino, diethylamino, di(1-methylethyl)amino, and the like.

“Heteroaryl” includes reference to a monovalent aromatic radical or group having one or more rings incorporating one, two or three heteroatoms within the ring (chosen from nitrogen, oxygen, or sulfur). These heteroaryls can optionally have hydrogen atoms substituted with one or more other substituents. Examples of these heteroaryl radicals include optionally substituted benzofurans, benzo[b]thiophene 1-oxide, indoles, 2- or 3-thienyls or thiophenyls, thiazoyls, pyrazines, pyridines.

The terms heteroalkyl, heteroalkylene, heteroalkenyl, heteroalkenylene, heteroalkynyl, and heteroalkylene include reference to alkyl, alkylene, alkenyl, alkenylene, alkynyl, and alkynylene radicals or groups, in which one or more of the carbon atoms have been replaced or substituted with atoms such as but not limited to single or multiply bonded nitrogen, sulfur, oxygen, or these atoms having one or more hydrogens to satisfy the valancy requirements of the atom. Such substitutions can be used to form molecules having functional groups including but not limited to amines, ethers, and sulfides. A non-limiting example of a heteroalkynyl group is illustrated by the group —CH(Me)OCH₂C═CH.

“Heterocycloalkyl radical” include reference to a monovalent saturated carbocyclic radical or group consisting of one or more rings, incorporating one, two or three heteroatoms (chosen from nitrogen, oxygen or sulfur), which can optionally be substituted with one or more substituents.

“Heterocycloalkenyl” includes reference to a monovalent unsaturated carbocyclic radical consisting of one or more rings containing one or more carbon-carbon double bonds where carbon atoms are replaced or substituted for by one, two or three heteroatoms within the one or more rings, the heteroatoms chosen from nitrogen, oxygen, or sulfur, the heterocycloalkenyl can optionally be substituted with one or more substituents.

Various groups used in the molecules of the present invention can have one or more hydrogens atoms substituted for chemical moieties or other substituents. Substituents may include but are not limited to halo or halogen (e.g, F, Cl, Br, I), haloalkyls such as CF₃, —CF₂CF₃, —CH₂CF₃ and the like, thioalkyl, nitro, optionally substituted alkyl, cycloalkyl, aralkyl, aryl, heteroaryls like benzofurans, indoles, thienyls, thiophenyls, thiazoyls, pyrazines, pyridines, alkoxy pyridine, hydroxy (—OH), alkoxy (—OR), aryloxy, alkoxyheteroaryl, cyano (—CN), carbonyl —C(═O)—, carboxy (—COOH) and carboxylate salts; —(CH₂)ₚC(═O)OH, groups or radicals —(CH₂)ₚC(═O)O(alkyl), and —(CH₂)ₚC(═O)NH₂ where p is independently the integer 0, 1, 2, or 3; sulfonates such as but not limited to tosyl, brosyl, or mesyl; sulfone, imine, or oxime groups, groups like —(C═O)O alkyl, aminocarbonyl or carbamoyl —(C═O)NH₂), —N-substituted aminocarbonyl —(C═O)NHR″, amino, alkylamino (—NHR″) and dialkylamino (—NHR′R″). In relation to the aforementioned amino and related groups, each moiety R′ or R″ can be, independently include of H, allyl, cycloalkyl, aryl, heteroaryl, aralkyl or optionally-substituted alkyl, cycloalkyl, aryl, heteroaryl, araalkyl.

In a further aspect, the invention provides a method of inhibiting apoptosis comprising administering the inventive compounds and compositions to a cell. For example, compounds according to the present invention can selectively bind to the FasR or the TNFR protein and inhibit cell death in the presence of FasL with EG₅₀ values in the nM range. The method can be employed on cells in vitro or in vivo. However, where employed in vivo, the application can be employed in human patients undergoing medical treatment or in animals (e.g., dogs, cats, horses, cows, pigs, sheep, goats, chickens, monkeys, rabbits, rats, mice, etc) undergoing veterinary care or laboratory study.

For medical or veterinary application, the invention provides a method of treating a Fas or TNFR-related disease comprising administering a therapeutically effective amount the inventive compound to a human or animal (typically mammalian) patient. The inventive method can be employed to treat a diseases and conditions associated with TNFR proteins, including, but not limited to sepsis, ischemia, reperfusion injury, acute or chronic liver dysfunction, cancer, chronic inflammatory disease and auto-immune disease.

In accordance with the inventive method, the compound according to the present invention is administered to the human or animal patient in an amount and at a location effective to treat the Fas or TNFR-related disease. “Administering” when used in conjunction with a therapeutic means to administer a therapeutic directly into or onto a target tissue or to administer a therapeutic to a patient whereby the therapeutic positively impacts the tissue to which it is targeted. “Administering” a composition may be accomplished by oral, rectal, topical, nasal, intradermal, inhalation, intra-peritoneal, or parenteral routes (i.e., subcutaneous, intravenous, intramuscular, or infusion), in combination with other known techniques.

An effective amount is that amount of a preparation that alone, or together with further doses, produces the desired response. This may involve only slowing the progression of the disease temporarily, although preferably, it involves halting the progression of the disease permanently or delaying the onset of or preventing the disease or condition from occurring. This can be monitored by routine methods. Generally, doses of active compounds would be from about 0.01 mg/kg per day to 1000 mg/kg per day. It is expected that doses ranging from 50-500 mg/kg will be suitable, preferably intravenously, intramuscularly, orally, or intradermally, and in one or several administrations per day.

In general, routine experimentation in clinical trials can help optimize specific ranges for optimal therapeutic effect for each therapeutic agent and each administrative protocol, and administration to specific patients will be adjusted to within effective and safe ranges depending on the patient condition and responsiveness to initial administrations. However, the ultimate administration protocol will be regulated according to the judgment of the attending clinician considering such factors as age, condition and size of the patient, the potencies, the duration of the treatment, and the severity of the disease being treated. For example, a dosage regimen of the inventive compound can be oral administration of from 1 mg to 2000 mg/day, preferably 1 to 1000 mg/day, more preferably 50 to 600 mg/day, in two to four (preferably two) divided doses, to reduce tumor growth. Intermittent therapy (e.g., one week out of three weeks or three out of four weeks) may also be used.

In the event that a response in a subject is insufficient at the initial doses applied, higher doses (or effectively higher doses by a different, more localized delivery route) may be employed to the extent that the patient tolerance permits. Multiple doses per day are contemplated to achieve appropriate systemic levels of compounds. Generally, a maximum dose is used, that is, the highest safe dose according to sound medical judgment. Those of ordinary skill in the art will understand, however, that a patient may insist upon a lower dose or tolerable dose for medical reasons, psychological reasons or for virtually any other reason.

A variety of administration routes are available. The particular mode selected will depend, of course, upon the particular chemotherapeutic drug selected, the severity of the condition being treated and the dosage required for therapeutic efficacy. The methods of the invention, generally speaking, may be practiced using any mode of administration that is medically acceptable, meaning any mode that produces effective levels of the active compounds without causing clinically unacceptable adverse effects. Such modes of administration include, but are not limited to, oral, rectal, topical, nasal, intradermal, inhalation, intra-peritoneal, or parenteral routes. The term “parenteral” includes subcutaneous, intravenous, intramuscular, or infusion. Intravenous or intramuscular routes are particularly suitable for purposes of the present invention.

The compounds of the present invention may include a pharmaceutically acceptable carrier. The term “pharmaceutically-acceptable carrier” as used herein means one or more compatible solid or liquid fillers, diluents, other excipients, or encapsulating substances which are suitable for administration into a human or veterinary patient. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner so as not to substantially impair the desired pharmaceutical efficacy. “Pharmaceutically acceptable” materials are capable of administration to a patient without the production of undesirable physiological effects such as nausea, dizziness, rash, or gastric upset. It is, for example, desirable for a therapeutic composition comprising pharmaceutically acceptable excipients not to be immunogenic when administered to a human patient for therapeutic purposes.

The pharmaceutical compositions may contain suitable buffering agents, including: acetic acid in a salt; citric acid in a salt; boric acid in a salt; and phosphoric acid in a salt. The pharmaceutical compositions also may contain, optionally, suitable preservatives, such as: benzalkonium chloride, chlorobutanol, parabens and thimerosal.

The pharmaceutical compositions may conveniently be presented in unit dosage form and may be prepared by any of the methods well known in the art of pharmacy. All methods include the step of bringing the active agent into association with a carrier that constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for parenteral administration conveniently comprise a sterile aqueous preparation of the inventive compound, which is preferably isotonic with the blood of the recipient. This aqueous preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa. which is incorporated herein in its entirety by reference thereto.

The delivery systems of the invention are designed to include time-released, delayed release or sustained release delivery systems such that the delivering of the inventive compound occurs prior to, and with sufficient time, to cause sensitization of the site to be treated. The inventive compound may be used in conjunction with other therapeutic agents or therapies. Such systems can avoid repeated administrations of the inventive compound, increasing convenience to the subject and the physician, and may be particularly suitable for certain compositions of the present invention.

Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drags are described in, for example, U.S. Pat. No. 5,075,109. Delivery systems also include non-polymer systems that are: lipids including sterols such as cholesterol, cholesterol esters and fatty acids or neutral fats such as mono-di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the active compound is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480. In addition, pump-based hardware delivery systems can be used, some of which are adapted for implantation.

Use of a long-term sustained release implant may be desirable. Long-term release, are used herein, means that the implant is constructed and arranged to deliver therapeutic levels of the active ingredient for at least 30 days, and preferably 60 days. Long-term sustained release implants are well-known to those of ordinary skill in the art and include some of the release systems described above.

This invention and embodiments illustrating the method and materials used may be further understood by reference to the following non-limiting examples.

### EXAMPLE 1

The present example illustrates the measured associated constants for various substitutions for inventive compounds.

Materials and Methods.

Cell Lines, Chemicals, Antibodies & Proteins. Recombinant Human Fas/TNFRSF6/Fc Chimera (cat no. 326-FS-050), Human Fas Ligand/TNFSF6 (cat. No. 126-FL), Anti-human Fas TNFRSF6 Polyclonal Antibody (cat. no. AF326) and Anti-6×Histidine Cross-Linking Ab (cat no. MAB050) were purchased from R&D Systems. Anti-human, IgM-HRP (cat. no. AP320P) was purchased from Chemicon International. Phosphate buffered saline (cat. no. P3563), sodium azide (cat. no. 71289) and 3,3′,5,5′-Tetramethylbenzidine (TMB) Liquid Substrate System for ELISA (cat. no. T0440) were purchased from Sigma-Aldrich. Cliniplate 96-well microplates w/enhanced binding (cat. no. 28298-604) were purchased from VWR. Costar, 96-well, cell culture assay plates (cat. no. 3632) were ordered from Corning Incorporated. Jurkat cells (ATCC no. CRL-1990) and L929 (ATCC no. CRL-2148) were purchased from ATCC and were propagated in RPMI 1640 medium with 2 mM L-glutamine, adjusted to contain 1.5 g/L sodium bicarbonate, 4.5 g/L glucose, 10 mM HEPES, and 1 mM sodium pyruvate, 90%; 10% fetal bovine serum. CellTiter Blue Cell Viability Assay (cat. no. TB317) was purchased from Promega.

ELISA. Peptides were dissolved in a coating buffer solution (0.05M Sodium Carbonate, 0.02% Sodium Azide, pH=9.6) at the appropriate concentrations to a final concentration of 10 μM. Dilutions were made accordingly to reach desired experimental concentrations. 100 μl aliquots of the peptide solution was added to each well and incubated for 2 hours. All incubations were carried out with shaking at T=37° C. Wells were washed by pipetting solution out of each well, adding 200 μL of washing solution (PBS-Tween; 0.5%), shaking for 15 minutes and emptying each well. Three wash cycles were carried out prior to addition of 200 μL of blocking solution (1% BSA in PBS buffer) to each well followed by incubation for 2 hours. Wells were then washed (as per above procedure) three times. Protein solutions of the appropriate concentrations (Vₛₒₗᵤₜᵢₒₙ 50 μL) were then added and incubated for 2 hours. After washing three times (as per above protocol) Fas specific antibody solution (˜1:20,000) was added to each well in 100 μL aliquots and incubated for 1 hour. After washing three times (as per above protocol) IgM-HRP solution (˜1:20,000) was added to each well in 100 μL aliquots and incubated (with shaking) for 1 hour at T=37° C. Wells were washed three times prior to the addition of ready-to-use TMD, followed by incubation for 30 minutes. 2M H₂SO₄ (3 drops) was added to stop colorimetric reaction and the absorption was taken at 450 nm on a Perkin-Elmer, Victor3 Model automated plate reader. Data was analyzed using standard regression fits in Excel.

Peptide Synthesis. Peptides were synthesized on an Advanced ChemTech, multi-well, solid-phase synthesizer using traditional F-moc chemistry. C-terminal amide functionalized peptides were generated from Rinlc Amide MBHA resin and C-terminal acid functionalized peptides were generated from pre-loaded, TGA Resins. All N-a-F-moc protected amino acids were purchased from Novabiochem. Peptides were purified by HPLC via reverse phase, C-18 column on a Beckman BioSys, model 510. Characterization was carried out by LC/MS on an Agilent 110 Series Trap spectrometer.

Apoptosis Assay. Jurkat cells in RPMI 1640 medium were diluted such that 100 μL of cell solution had an overall cell count of 2×10⁴ cells per well. Control reactions were diluted with sterile PBS buffer to final volume of 200 μL per well. Peptide solutions (in PBS buffer, pH=7.3) were added in appropriate volumes to reach the desired concentrations in each well given a total volume of 200 μL per well. Apoptosis was induced by the addition of cross-linked human FasL at a concentration of 0.5 ng/ml. Plates were then incubated under sterile conditions, with shaking in a Kendro, model Hera Cell 150 incubator at T=37° C., % CO₂=5 for 3 hours. A Cell Titer Blue Cell Viability Assay kit was used (as per accompanying protocol) to determine % apoptosis in cells. Fluorescence readings (560/590 nm) were taken after 4 hours under above cited incubation conditions on a Perkin-Elmer, Victor3 Model automated plate reader. Data was analyzed using standard regression fits in Excel.

Results. Initial studies with a-Met revealed that only the first 106 amino acids of the N-terminal were essential for effective binding with FasR. Furthermore, upon a direct sequence alignment with FasL, as depicted below, it was shown that only a short, ten amino acid stretch was shown to exhibit similar homology and of that, only a tetrameric portion was fully conserved; namely, the YLGA sequence.

Sequence Alignment Data for A) Binding Regions of a-Met and FasL B) Homologous Proteins from the TNF Superfamily.

(SEQ ID NO:38)

αMet: PIQ NVV LHK HHI YLG ATN YIY VLN DKD LQK

(SEQ ID NO:39)

FasL: YCT TGQ MWA RSS YLG AVF NLT SAD HLY VNV

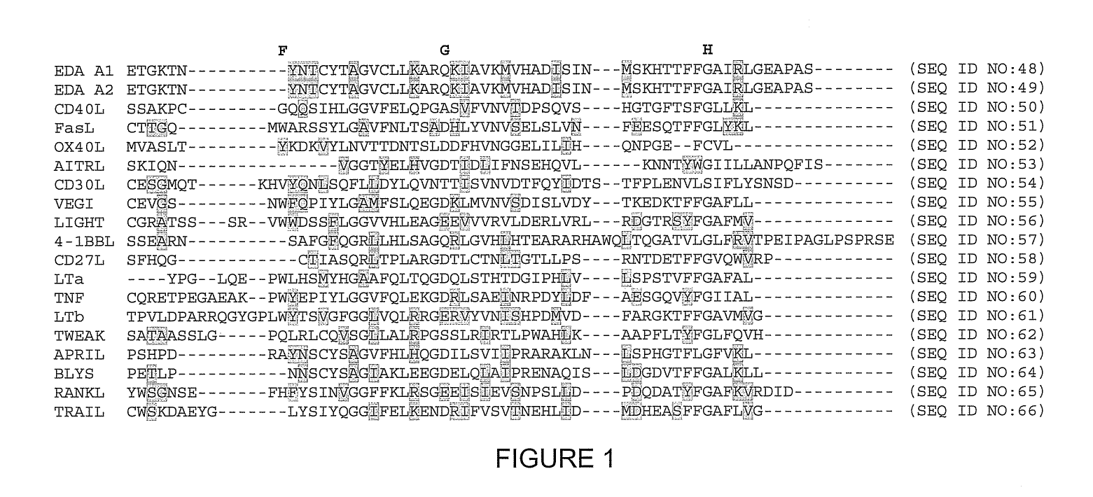

As depicted in FIG. 1, a comparison of this four amino acid sequence with other TNF superfamily ligands reveals that a similar motif suggesting that this may provide the critical binding energy required for ligand/receptor association in this family of proteins. Previously conducted studies have shown that a synthetic tetrapeptide exhibited only a modest difference in Kₐ values from a longer dodecamer sequence. These findings suggested the desirability of looking in depth at the structure-function relationship surrounding the primary binding sequence by systematic replacement of each residue in the tetramer with alternate amino acids. An examination of the effect of N- or C-terminal derivatization, as well as N-methylation of the amide bonds, provides information regarding the optimal binding sequence and possibilities for the development of peptidomimetic compounds having formula VI, as shown below:

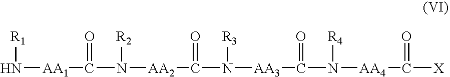

wherein AAₙ is an amino acid and Rₙ is an allyl group, and X is selected from OH and NH₂.

Effect of Amino Acid Substitution on Binding Affinities. The binding affinity of each tetrapeptide was determined by standard ELISA with a concentration range of 1 nM to 1 μM. In the first series of peptides, alterations were made only to the tyrosine residue while retaining the final three amino acids homologous in the parent sequence (LGA). The results of these changes are listed in Table 1.

TABLE 1

Measured Association Constants (Ka) for YLGA
(SEQ ID NO:1) to XLGA Substituted Peptides

| | | | |
|---|---|---|---|
| Residue | Ka(M⁻¹) | Residue | Kₐ(M⁻¹) |

| | | | |
|---|---|---|---|
| YLGA | 2 × 10⁸ | LLGA | 1 × 10⁷ |
| (SEQ ID NO:1) | | (SEQ ID NO:40) | |
| SLGA | 2 × 10⁸ | ILGA | 9 × 10⁶ |
| (SEQ ID NO:13) | | (SEQ ID NO:41) | |
| TLGA | 2 × 10⁸ | KLGA | 8 × 10⁶ |
| (SEQ ID NO:14) | | (SEQ ID NO: 47) | |
| FLGA | 1 × 10⁸ | MeO-FLGA | 8 × 10⁵ |
| (SEQ ID NO:15) | | (SEQ ID NO:15) | |
| WLGA | 8 × 10⁷ | [symbol] F-FLGA | 8 × 10⁵ |
| (SEQ ID NO:16) | | (SEQ ID NO:15) | |
| DLGA | 5 × 10⁷ | ALGA | no binding |
| (SEQ ID NO:17) | | (SEQ ID NO:42) | observed |
| ELGA | 5 × 10⁷ | GLGA | no binding |
| (SEQ ID NO:18) | | (SEQ ID NO:43) | observed |

Studies with the methoxyphenylalanine and p-fluorophenylalanine, highlighted the importance of the hydroxy moiety located on the tyrosine residue as evidenced by the 2.5 order of magnitude drop off in binding affinities between the YLGA (SEQ ID NO:1) and the substituted FLGA (SEQ ID NO:15) sequences. The presence of an aromatic residue at this first position was seen to contribute substantially less to the overall binding energy than the presence of an OH functionality within the site. This is particularly noticeable when contrasting the binding affinities of the aliphatic side chains (A, L, I) versus their hydroxy containing counterparts (S, T). Substitution for an alanine (—CH₃) in the first position resulted in a complete loss of binding (within the limits of detection) while the serine group (—CH₂OH) showed (within margin of error) identical binding to the original tyrosine peptide. The possibility of this observation resulting exclusively from polarity effects is argued against by the comparison of the acid containing residue substitutions (D, E) which exhibited a four fold drop off upon introduction of the acid functional groups.

Substitutions at the second position revealed a small degree of flexibility when substituting the leucine residue for other members of the aliphatic side chain group (V, I). In particular, the YAGA (SEQ ID NO:10) sequence showed a twenty percent higher Kₐ value than the parent YLGA (SEQ ID NO:1) sequence (see Table 2), suggesting that a shorter, less hindered sequence may be favorable for optimal binding. Examination of the YGGA (SEQ ID NO:44) peptide reveals that hydrophobic interactions contribute to binding affinity, as elimination of the methylene unit from the side chain results in a 20-fold decrease in measured Kₐ. This is further supported by the complete loss of binding when threonine, a polar residue, replaces the non-polar leucine. Steric factors must also be considered as demonstrated by the decrease in binding affinities with the branched aliphatic chains (V, I) and the complete loss of binding with the aromatic phenylalanine group.

TABLE 2

Measured Association Constants (Ka) YLGA
(SEQ ID NO:1) and YXGA Substituted Proteins

| | | | |
|---|---|---|---|
| Residue | Kₐ(M⁻¹) | Residue | Kₐ(M⁻¹) |

| | | | |
|---|---|---|---|
| YLGA | 2 × 10⁸ | YGGA | 1 × 10⁷ |
| (SEQ ID NO:1) | | (SEQ ID NO:44) | |
| YAGA | 3 × 10⁸ | YFGA | no binding |
| (SEQ ID NO:10) | | (SEQ ID NO:45) | observed |
| YVGA | 2 × 10⁸ | YTGA | no binding |
| (SEQ ID NO:11) | | (SEQ ID NO:46) | observed |
| YIGA | 2 × 10⁸ | | |
| (SEQ ID NO:12) | | | |

The remaining two positions showed remarkably little ability to accommodate any substitution within the sequence, as shown in Tables 3 and 4. Replacement of the glycine residue with the larger, aliphatic side chain amino acids alanine or leucine, saw a 20-fold decrease in Kₐ while the more sterically encumbered isoleucine or d-alanine showed no observable binding. Similarly, substitution of the terminal alanine by either a smaller side chain (G) or larger ones (L, I) exhibited nearly 10 times less affinity for FasR binding as the original YLGA sequence (SEQ ID NO:1). This overall limited flexibility has direct implications for the design of biologically active peptidomimetics.

TABLE 3

Measured Association Constants (Ka) for YLGA
(SEQ ID NO:1) and YLXA Substituted Peptides

| | |
|---|---|
| Residue | Kₐ(M⁻¹) |

| | |
|---|---|
| YLGA | 2 × 10⁸ |
| (SEQ ID NO:1) | |
| YLAA | 1 × 10⁶ |
| (SEQ ID NO:6) | |
| YLLA | 1 × 10⁶ |
| (SEQ ID NO:7) | |
| YLIA | no binding observed |
| (SEQ ID NO:8) | |
| YldAA | no binding observed |
| (SEQ ID NO:9) | |

TABLE 4

Measured Association Constants (Ka) for YLGA
(SEQ ID NO:1) and YLGX Substituted Peptides

| | |
|---|---|
| Residue | Ka(M − 1) |

| | |
|---|---|
| YLGA | 2 × 10⁸ |
| (SEQ ID NO:1) | |
| YLGG | 4 × ⁷ |
| (SEQ ID NO:2) | |
| YLGL | 3 × 10⁷ |
| (SEQ ID NO:3) | |
| YLGI | 3 × 10⁷ |
| (SEQ ID NO:4) | |
| YLGT | no binding observed |
| (SEQ ID NO:5) | |

Effect of N-Terminal, C-Terminal and Amide Linkage Alterations on Binding Affinities. The ability to methylate the amide nitrogens is of interest in terms of designing peptides with more pharmacologically favorable characteristics. Consistent with the above findings, methylation of the amide bond, prior to the G or the A position, results in a no observable binding of the peptide to the receptor (R₃, R₄ in Formula V). Alkylation at the R₁ or R₂ positions, however, showed no significant decrease in biding affinity suggesting that these positions are less intricately coupled with the binding domain. The effect of replacing the amide functionality at the C-terminal for an acid group also showed little effect on the overall binding ability of the peptide to bind the FasR protein.

Correlating the In-vitro Binding Assays with Cellular Activity. FasL activated apoptosis is known to occur by binding of the ligand to the receptor thereby initiating a series of signaling events leading to caspase production and subsequent cell death. If the YLGA (SEQ ID NO:1) motif isolated in both the α-Met and FasL proteins, is indeed the primary sequence responsible for binding to FasR then at a given concentration, peptides of sufficient Kₐ values should preferably bind to the receptor. This in turn would block the binding site from FasL and inhibiting cell death in the presence of both peptide and FasL. In order to examine this possibility, Jurkat cells (known to upregulate the Fas receptor) were treated with varying concentrations of peptide and then exposed to recombinant FasL. The overall cell viability after an incubation period of 2 hours, for a representative number of peptides shows a direct correlation between the dissociation constant of the peptide and the measured EC₅₀ value, as shown in Table 5.

TABLE 5

Measured EC₅₀ Values for Various
Peptides and Corresponding K_{D} Values

| | | | |
|---|---|---|---|
| Peptide | Kₐ(M⁻¹) | K_{D}(nM) | EC₅₀ (nM) |

| | | | |
|---|---|---|---|
| YAGA | 3 × 10⁸ | 4 | 6 |
| (SEQ ID NO:10) | | | |
| YLGA | 2 × 108 | 5 | 6 |
| (SEQ ID NO:1) | | | |
| SLGA | 2 × 108 | 5 | 6 |
| (SEQ ID NO:13) | | | |
| FLGA | 1 × 108 | 10 | not measured |
| (SEQ ID NO:15) | | | |
| WLGA | 8 × 107 | 13 | no |
| (SEQ ID NO:16) | | | inhibition |
| | | | observed |
| YLGG | 4 × 107 | 28 | 52 |
| (SEQ ID NO:2) | | | |
| LLGA | 1 × 10⁷ | 88 | 93 |
| (SEQ ID NO:40) | | | |
| YLAA | no observable | — | no inhibition |
| (SEQ ID NO:6) | binding | | observed |
| LGA (trimer) | no observable | — | no inhibition |
| | binding | | observed |

This correlation implies a peptide-protein interaction mechanism, wherein whereby tightly bound peptides are capable of blocking FasL association with the receptor and as such, inhibit FasL induced apoptosis. A sequence alignment of the corresponding binding regions of other members of the TNF ligand superfamily, reveals similar motifs, as shown in FIG. 1. In cases where the YLGA (SEQ ID NO: 1) motif is essentially conserved, such as TNF, preliminary results suggest the ability to selectively inhibit one pathway over another by simple modifications of the amino acid sequence, as depicted in Table 6.

TABLE 6

Selectivity for FasR vs. TNFR
via Change in Final Residue

| | |
|---|---|
| TNFR | FasR |

| | | |
|---|---|---|
| YLGA K_{d} | 6 nM | 5 nM |
| (SEQ ID NO:1) | | |
| EC₅₀ | 11 nM | 6 nM |
| YLGG K_{d} | 4 nM | 28 nM |
| (SEQ ID NO:2) | | |
| EC₅₀ | 8 nM | 52 Nm |

Effect of Flanking Residues on Binding Affinities. The protein Met was originally shown to require its first 106 amino acids of the alpha subunit in order to achieve binding to the FasR site. While this work has shown that tetrapeptides based from the original protein sequence can inhibit the FasL/FasR cell death pathway at low nM concentrations, the ability to improve upon this was examined by looking at the extended sequence on both the N-terminal and C-terminal side of the YLGA (SEQ ID NO:1) motif. Peptides up to 12 amino acids in length were synthesized based on the α-Met sequence and their Kₐ values measured for the Fas receptor, Table 7. While overall binding ability was enhanced by 4-14% over the YAGA sequence (SEQ ID NO:10), synthetic complexity and increased solubility factors make the increase in Kₐ of less utility for subsequent experiments than might otherwise be expected.

TABLE 7

Measured KD Values for
Elongated Peptide Sequences

| | |
|---|---|
| Peptide | Kₐ (M⁻¹) |

| | |
|---|---|
| HHI YLG ATN YIY | 3 × 10⁸ |
| (SEQ ID NO:29) | |
| HI YLG ATN YIY | 3 × 10⁸ |
| (SEQ ID NO:30) | |
| I YLG ATN YIY | 3 × 10⁸ |
| (SEQ ID NO:31) | |
| YLG ATN YIY | 3 × 10⁸ |
| (SEQ ID NO:32) | |
| HHI YLG ATN YI | 3 × 10⁸ |
| (SEQ ID NO:33) | |
| HHI YLG ATN Y | 3 × 10⁸ |
| (SEQ ID NO:34) | |
| HHI YLG ATN | 3 × 10⁸ |
| (SEQ ID NO:35) | |
| HHI YLG AT | 3 × 10⁸ |
| (SEQ ID NO:36) | |
| HHI YLG A | 3 × 10⁸ |
| (SEQ ID NO:37) | |

As shown in Table 8 below, the dissociation constant and the measured EC₅₀ values of the various peptides for Fas and TNFR1 were measured.

TABLE 8
Measured KD and EC50 Values for Fas and TNFR1

| | Fas | | TNFR1 | |
|---|---|---|---|---|
| Sequence | K_{d} | EC₅₀ | K_{d} | EC₅₀ |

| | | | | |
|---|---|---|---|---|
| YLGA | 4.5 | 11.0 | 5.7 | 6.1 |
| (SEQ ID NO:1) | | | | |
| YLGAVF | 1.12 | 3.05 | 4.80 | 6.43 |
| (SEQ ID NO:19) | | | | |
| SYLGA | 4.14 | 5.12 | 5.13 | 7.11 |
| (SEQ ID NO:20) | | | | |
| SSYLGA | 3.76 | 5.79 | 6.54 | 8.06 |
| (SEQ ID NO:21) | | | | |
| RSSYLGA | 1.27 | 3.28 | not | no inhibition |
| (SEQ ID NO:22) | | | measurable | |
| RSSYLGAVF | 0.91 | 3.16 | not | no inhibition |
| (SEQ ID NO:23) | | | measurable | |

The general conclusions drawn from the structure-function studies described above are summarized schematically below:

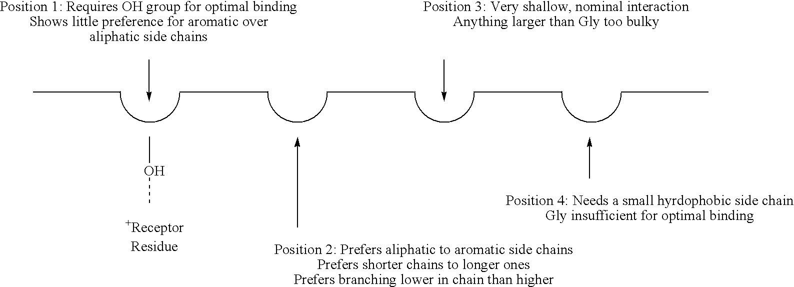

In general, these indicate a very selective binding pocket that is nearly optimized within the natural YLGA sequence (SEQ ID NO:1). Several results are of key importance with regard to designing future peptidomimetic compounds for use as therapeutic targets in Fas related diseases. First, the necessity for a hydroxyl group in the first position, suggests the presence of a positively charged residue or hydrogen bonding residue in the correlating position on the receptor. This result is consistent with a binding site structure proposed from modeling studies wherein it is suggested that R87 plays a critical role in strong FasL/FasR interactions via interaction with a tyrosine residue. The structure further proposes the presence of two key lysine groups as well as a secondary arginine within close proximity to the ligand; all of which could play a role in providing important binding interactions for the hydroxyl moiety on the ligand group. Also noteworthy is the key role the OH group plays in ‘anchoring’ the peptide to the binding site, as evidenced by the near complete loss of binding of the analogous, Y-free, trimer peptide, Table 4. This suggests that any synthetic modifications made in the development of a peptidomimetic compound would need to incorporate a similar motif.

Another observation is the enhanced binding ability garnered by replacement of the leucine at the second position with its smaller, aliphatic counterpart, alanine. In altering the side chain from a 3-carbon, terminally branched to a single carbon alkyl chain, the Kₐ value was increased by ˜20%. Yet, substitution to a glycine resulted in loss of binding, most likely due to reduced hydrophobic interactions between the peptide and the receptor. As such, it may be possible to further optimize this position by introducing a non-natural amino acid of intermediate length. This flexibility also extends to the amide bond of the residue where it was shown that methylation of the amide nitrogen resulted in only marginal loss of binding affinity making it a viable target for chemical modification in order to maximize the likelihood of biological activity. The inflexibility of both the third and fourth amino acids has direct implications for the design of biologically active peptidomimetics. In particular, the necessity for a small, non-interacting portion at the third position could allow for easy introduction of simple alkyl linkage in lieu of the peptide moiety thereby reducing the compounds overall susceptibility to proteolytic cleavage. In conjunction with C- and N-terminal derivatization, these factors combine to allow for the construction of a unique and specific target compound, optimized for the Fas receptor.

It should be noted that in examining the effect of flanking sequences on Kₐ for FasR, it was the α-Met structure that was utilized as a model rather than the FasL. A closer examination of the two proteins sequences reveals significant differences in polarity, size and charge of the flanking residues, as shown in FIG. 1. Thus, it is possible that extension of the peptide length to incorporate components from the FasL sequence may result in enhanced binding affinity of the peptides for the receptor. These studies are currently underway.

The interaction of FasR and its ligand, FasL is conserved across numerous species and yet little is known about the interaction on a molecular level. Several model studies and numerous biochemical approaches have been used in an attempt to better understand the structural components involved in this protein-protein interaction. More recently, studies have been carried out using peptides in order to further elucidate the mechanism of action of Fas induced cell death. In these studies, emphasis was placed on mimicking the FasL interaction in order to induce cell death or on targeting the receptor in such a way as to allow for FasL binding but inhibit receptor trimerization. Both instances, while useful in providing information regarding the disruption of the Fas pathway, provided no further structural information surrounding the actual binding interaction such that an antagonist to FasL could then be developed.

In this example, the various alterations carried out on the YLGA (SEQ ID NO:1) peptide have allowed for a better understanding of the binding pocket involved in the FasL/FasR interaction. Peptide structure was shown to control the tight coupling of binding affinity of a peptide for FasR with their demonstrated ability for cellular antagonism. These studies introduce a novel means of targeting the disruption of the TNFR cell death pathway, including the FasL/FasR pathway, and the development of therapeutic targets for Fas or TNFR-related disorders.

Based upon the foregoing, the compounds of the formula VII

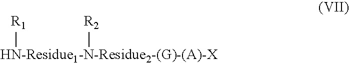

wherein Residue₁ is selected from Y, S, T, D, and E; Residue₂ is selected from A, V, L, I, M and methyl, ethyl, propyl, butyl and pentyl; R₁ is selected from methyl, ethyl, propyl, butyl, pentyl, hexyl, heptyl, octyl, nonyl, decyl, carboxylic acid, and an extended peptide up to three residues long selected from H—H—I, R—S—S, and E-P—I; R₂ is selected from methyl, ethyl, proply, butyl, pentyl, hexyl, heptyl, octyl, nonyl, decyl and carboxylic acid; and X is selected from COOH and CONH₂ have been shown to be effective binders for the FasR protein and act as effective FasL antagonists in the prevention of Fas induced apoptosis. The ability to vary the amino acid residues, R1 and R2 and X, in particular, presents a possible means for pharmacological optimization in the form of peptidomimetic derivatives.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A compound comprising the formula X1-X2-X3-X4, wherein
X1 is Y, S, T, F, W, D or E or a mimetic of Y, S, T, F, W, D, or E;
X2 is L, A, V, I, G, F, or T or a mimetic of L, A, V, I, G, F, or T;
X3 is G, A, L, I or A or a mimetic of G, A, L, I, or A; and
X4 is A, G, L, I, or T or a mimetic of A, G, L, I or T.

2. The compound of claim 1, wherein
X1 is Y, S, T, F, W, D or E,
X2 is L, A, V, I, G, F, or T,
X3 is G, A, L, I or A, and
X4 is A, G, L, I, or T.

3. A composition comprising the compound of claim 1 and a pharmaceutically acceptable carrier.

4. A compound comprising the formula X1-X2-X3-X4-X5-X6-X7-X8 wherein
X1 is Y, S, T, F, W, D or E or a mimetic of Y, S, T, F, W, D, or E;
X2 is a spacer;
X3 is L, A, V, I, G, F, or T or a mimetic of L, A, V, I, G, F, or T;
X4 is a spacer;
X5 is G, A, L, I or A or a mimetic of G, A, L, I, or A;
X6 is a spacer;
X7 is A, G, L, I, or T or a mimetic of A, G, L, I or T; and
X8 is a spacer.

5. The compound of claim 4, further comprising X0, wherein X0 is A1-A2-A4, wherein A1 is H, K, R, E or D or a mimetic of H, K, R, E, or D; A2 is S, A, or T or a mimetic of S, A, or T; and A3 is S, A, T, I, or V or a mimetic of S, A, T, I, or V.

6. A composition comprising a compound of claim 4 and a pharmaceutically acceptable carrier.

7. A composition comprising a compound of claim 5 and a pharmaceutically acceptable carrier.

8. A compound comprising the formula X1-X2-X3-X4-X5-X6-X7-X8 wherein
X1 is a spacer;
X2 is Y, S, T, F, W, D or E or a mimetic of Y, S, T, F, W, D, or E;
X3 is a spacer;
X4 is L, A, V, I, G, F, or T or a mimetic of L, A, V, I, G, F, or T;
X5 is a spacer;
X6 is G, A, L, I or A or a mimetic of G, A, L, I, or A;
X7 is a spacer; and
X8 is A, G, L, I, or T or a mimetic of A, G, L, I or T.

9. A composition comprising the compound of claim 8 and a pharmaceutically acceptable carrier.

10. A compound comprising the formula 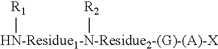
wherein Residue₁ is selected from Y, S, T, D, and E;
Residue₂ is selected from A, V, L, I, M, and an alkyl of 1 to 5 carbon atoms;
R₁ and R₂ are independently selected from H, an alkyl of 1 to 10 carbon atoms and COOH; and
X is selected from COOY and CONH₂.

11. A composition comprising the compound of claim 10 and a pharmaceutically acceptable carrier.

12. A compound comprising the formula 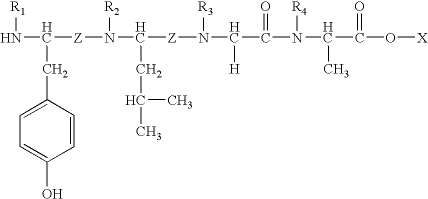
wherein Z₁ and Z₂ are independently selected from C═O and CH₂; R₁ is selected from a H, a lower allyl preferably of 1 to 10 carbon atoms, optionally a substituted lower alkyl, a carboxylic acid and an extended peptide up to three residues long selected from H—H—I, R—S—S, and E-P—I; R₂, is selected from a H, a lower alkyl, preferably of 1 to 10 carbon atoms, optionally a substituted lower alkyl, and a carboxylic acid; R₃, and R₄ are H, and X is selected from COOH and CONH₂.

13. A composition comprising the compound of claim 12 and a pharmaceutically acceptable carrier.

14. A method of inhibiting apoptosis comprising administering an effective amount of the composition of claim 3.

15. A method of inhibiting apoptosis comprising administering an effective amount of the composition of claim 6.

16. A method of inhibiting apoptosis comprising administering an effective amount of the composition of claim 9.

17. A method of inhibiting apoptosis comprising administering an effective amount of the composition of claim 11.

18. A method of inhibiting apoptosis comprising administering an effective amount of the composition of claim 13.

19. A method of treating a Fas or TNFR-related condition in a subject, comprising administering to said subject a therapeutically effective amount of the composition of claim 3.

20. The method of claim 16, wherein said Fas or TNFR-related condition is selected from the group consisting of sepsis, ischemia, reperfusion injury, and acute or chronic liver dysfunction, cancer, chronic inflammatory disease and auto-immune disease.

21. A method of treating a Fas or TNFR-related condition in a subject, comprising administering to said subject a therapeutically effective amount of the composition of claim 6.

22. The method of claim 21, wherein said Fas or TNFR-related condition is selected from the group consisting of sepsis, ischemia, reperfusion injury, and acute or chronic liver dysfunction, cancer, chronic inflammatory disease and auto-immune disease.

23. A method of treating a Fas or TNFR-related condition in a subject, comprising administering to said subject a therapeutically effective amount of the composition of claim 9.

24. The method of claim 23, wherein said Fas or TNFR-related condition is selected from the group consisting of sepsis, ischemia, reperfusion injury, and acute or chronic liver dysfunction, cancer, chronic inflammatory disease and auto-immune disease.

25. A method of treating a Fas or TNFR-related condition in a subject, comprising administering to said subject a therapeutically effective amount of the composition of claim 11.

26. The method of claim 25, wherein said Fas or TNFR-related condition is selected from the group consisting of sepsis, ischemia, reperfusion injury, and acute or chronic liver dysfunction, cancer, chronic inflammatory disease and auto-immune disease.

27. A method of treating a Fas or TNFR-related condition in a subject, comprising administering to said subject a therapeutically effective amount of the composition of claim 13.

28. The method of claim 27, wherein said Fas or TNFR-related condition is selected from the group consisting of sepsis, ischemia, reperfusion injury, and acute or chronic liver dysfunction, cancer, chronic inflammatory disease and auto-immune disease.
